(19) United States
(12) Patent Application Publication
(10) Pub. No.: US 2010/0217079 A1
(43) Pub. Date: Aug. 26, 2010

(54) Endoscopic Capsule

(76) Inventor: Peter Tichy, Uttenreuth (DE)

Correspondence Address: BRINKS HOFER GILSON & LIONE, P.O. BOX 10395, CHICAGO, IL, 60610, US

(21) Appl. No.: 12/706,569

(22) Filed: Feb. 16, 2010

(30) Foreign Application Priority Data
Feb. 19, 2009 (DE) DE 102009009616.7

(51) Int. Cl. A61B 1/00

(52) U.S. Cl. 600/118

(57) ABSTRACT

A capsule for endoscopic examinations and a method for assisting the advancement of the capsule through organs are provided. In addition to a device for advancing the capsule through an organ under investigation, the capsule is also provided with a device for generating movements of the capsule to reduce the edge friction impeding the advancement of the capsule. The device for generating movements of the capsule is activated using electromagnetic radiation irradiated from outside to a receiving system of the capsule. The device generates a movement, which helps overcome inhibiting frictional forces.

The present patent document claims the benefit of DE 10 2009 009 616.7, filed Feb. 19, 2009, which is hereby incorporated by reference.

### BACKGROUND

The present embodiments relate to a capsule for use in endoscopic examinations.

Classical endoscopy is a widely established method in medicine, both for examining or diagnosing, as well as for treating or administering therapy to a patient. In classical endoscopy, an endoscope or a catheter is introduced into a hollow organ of the patient (e.g., the stomach or the intestine) via a bodily orifice of the patient (e.g., the mouth or anus).

Conventional endoscopes do, however, have disadvantages. For example, conventional endoscopes have a limited range extending from the bodily orifice to the interior of the body of the patient or a limited flexibility when it comes to following curves or loops of hollow organs.

The small intestine of a patient may have a length of 7 to 8 m and is, for example, not fully accessible using a conventional endoscope with a limited range or limited flexibility.

Endoscopy systems employing magnetically controlled endoscopic capsules (e.g., endorobots) have been proposed to allow better investigation over the entire length of the intestinal tract. A magnetically controlled endoscopic capsule is described in DE 101 42 253 C1, for example. Magnetic guidance is achieved using magnetic forces that result from magnetic gradient fields that act on a permanent magnet in the capsule, the magnetic gradient field being generated by using an external guidance magnet. The external guidance magnet is an electromagnet such as is described, for example, in DE 103 40 925 B3 or WO 2006/092421 A1. In another embodiment, the guidance magnet includes one or more mechanically movable permanent magnets. As an alternative to magnetic guidance using magnetic forces, the capsule can, as described in US 2003/0181788 A1, be provided externally with a kind of thread and moved according to the principle of an Archimedes screw through a section of the intestine, while magnetic torques that are produced due to the interaction of a rotating external magnetic field with a permanent magnet fixedly incorporated into the capsule act on the capsule. The magnetization direction of the permanent magnet of the capsule may lie normal to the longitudinal axis of the capsule. The position and orientation of the capsule can be measured partially electromagnetically, as described, for example, in WO 2005/120345 A2.

Typically, the endorobot is navigated using a force input device, (e.g., a 6D mouse). The gradient direction, which corresponds to the superposition of the three individual systems, can be determined by tilting an input lever forward/back and right/left, as well as by pressing or lifting the input lever; the amplitude can be determined by turning the input lever. The forces applied to the input device may be proportional to the force applied to the instrument.

When performing methods in capsule endoscopy, obstacles may be created due to the position of the patient such that there are intestinal loops in a section of the intestine lying in a way that cannot be overcome by the endoscopic capsule or can be overcome only with great difficulty. Such obstacles include, for example, kinks in the intestine, very tight curves, polyps, or the compression of portions of the intestine due to organs lying on the intestine (e.g., other intestinal loops). The rubbing of the capsule against the interior wall of body cavities may lead to problems with movement or to blockages of movement. The problems with movement and blockages of movement can be removed by application of proportionally great magnetic forces onto the capsule, which constitutes a very complex and involved solution.

### SUMMARY AND DESCRIPTION

The present embodiments may obviate one or more of the drawbacks or limitations inherent in the related art. For example, in one embodiment, the movement of an endoscopic capsule during the examination of patients may be improved.

The present embodiments may provide, in addition to the advancement of the capsule with the aid of an advancing device (e.g., by using an integrated magnet and external magnetic fields), the generation of a movement through which obstructions (e.g., severe edge friction or jamming of the capsule) in the course of the advancement or navigation of the capsule through organs may be counteracted more effectively. The advancement of the capsule, with the aid of the advancing device, is facilitated in the event of movement-inhibiting edge friction or edge contact occurring. The generation of the movement may also assist the capsule to overcome inhibiting frictional forces.

The movement may include, for example, a jerking, a vibrating, a pulsating or an oscillating action, thereby increasing the freedom of movement of the capsule (e.g., as a result of the induced lessening of the friction with organ walls) and a further advancement with the aid of the advancing device.

The movement may be situationally triggered (e.g., when an obstruction of the capsule occurs). Parameters of the advancing device, for example, may be used as a criterion for the situational triggering of the movement. In one embodiment, the forces to be applied for the advancement with the aid of the advancing device (e.g. magnetic forces) may be used as a criterion for triggering or activating the movement. In one embodiment, the criterion may include a predefined maximum force not being able, or no longer being able to move the capsule a defined extent (e.g., a minimum speed or distance).

If a path through an organ under investigation or the position of the capsule in the organ is visualized externally (e.g., outside of the patient under examination) so that the capsule may be controlled by the operating personnel, then a decision concerning an activation of the movement may be made on the basis of the visualization or on the basis of an evaluation of optical information transmitted by the capsule. The operating personnel can see (e.g., on a monitor) that the capsule is not moving forward as desired and can activate an additional movement of the capsule to reduce the frictional forces acting on the capsule.

Manual or automatic activation is possible. Manual activation may also be provided in addition to automatic activation.

In addition to the advancing device for conveying the capsule through an organ under investigation, the capsule according to the present embodiments is provided with a device for generating movements to reduce edge friction or edge contact impeding the advancement of the capsule.

In one embodiment, the device is configured for generating movements, for example, using an ultrasonic resonator, a bobbin arranged in a coil, or an unbalanced motor. In one embodiment, the device for generating movements may use the physical effect of magnetostriction or electrostriction. In one embodiment, the capsule walls may be configured to generate movement using the effect of magnetostriction or electrostriction.

The activation of the device for generating movements or the triggering of the generation of a movement by the device is effected using an external (e.g., initiated from outside the patient under examination) irradiation of electromagnetic radiation.

In one embodiment, the irradiation of the electromagnetic radiation may directly cause energy to be supplied to the device for generating movements. In other words, the irradiated radiation represents energy that quickly feeds the device for generating movements. In one embodiment, the length of time during which the device will generate movements may be specified using the period of time the irradiation lasts. Thus, for example, a criterion for terminating the generation of movements may be specified (e.g., analogously to a criterion for the activation, using forces to be applied or an external visualization of the advancement or position of the capsule). Upon the criterion being fulfilled, the irradiation will be terminated, the energy supply to the capsule will be cut off, and the additional movement generation will be terminated.

In one embodiment, the capsule may include an energy store (e.g., a battery). The electromagnetic radiation irradiated for activation purposes represents a signal through which a supply of energy from the energy store to the device for generating movements is effected or triggered. In one embodiment, the irradiation of a second signal will stop the device for generating movements or terminate the supplying of energy from the energy store. In one embodiment, the energy store is configured for being charged using energy transmitted wirelessly from an external source.

Other combinations of energy supply to and activation of the device for generating movements may be found. For example, in one embodiment, an external irradiation of energy may be provided to supply the device for generating movements with energy (e.g., additional energy) only in a specific mode (e.g., boost mode) that is provided for overcoming obstructions during the advancement of the capsule. In another mode, the irradiated energy will be used, for example, for supplying energy to other parts of the capsule. The switching between modes may be effected using externally transmitted control signals.

### BRIEF DESCRIPTION OF THE DRAWINGS

FIG. 1 shows an endoscopic capsule,

FIG. 2 shows the navigation of an endoscopic capsule through an intestinal system,

FIG. 3 shows one embodiment of an endoscopic capsule having a device for generating vibrations,

FIG. 4 shows one embodiment of an endoscopic capsule having a device for generating vibrations,

FIG. 5 shows one embodiment of an energy supply to a device for generating vibrations,

FIG. 6 shows one embodiment of an energy supply to a device for generating vibrations.

### DETAILED DESCRIPTION

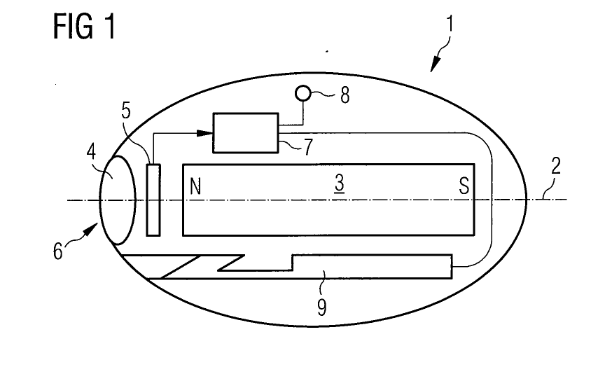

FIG. 1 shows an endoscopic capsule as described in DE 101 42 253 C1 (e.g., an endorobot).

A capsule 1 has an ellipsoid-shaped housing in which a bar magnet 3 is aligned collinearly to a principal axis 2. A video camera 6 may include a lens 4 and a CD sensor 5, and records images, which are transmitted externally using an RF transmitter 7 and an antenna 8. Different measuring instruments, biopsy instruments or treatment instruments may also be controlled via radio (e.g., via the antenna 8). As shown in FIG. 1, one embodiment may include a biopsy pistol 9 controlled via the antenna 8.

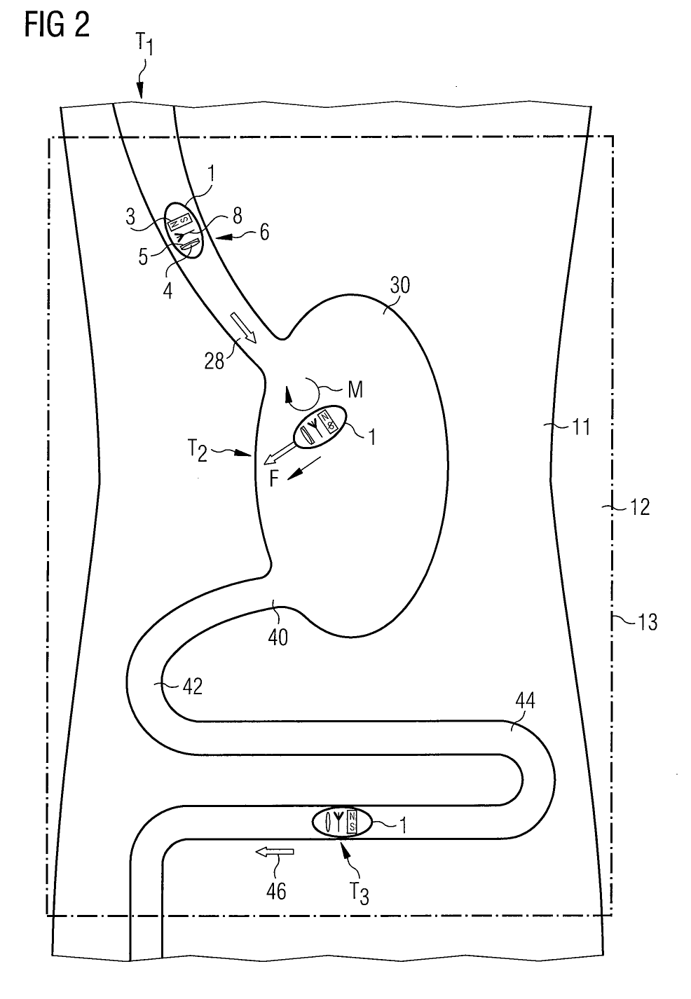

FIG. 2 shows the capsule 1 shown in FIG. 1 in action. FIG. 1 schematically illustrates a patient 11 who has been brought into a working room 12 of a magnetic coil system 13. A capsule endoscopy is to be performed on the patient 11. An endoscopic capsule 1 is therefore administered orally to the patient 11. The capsule 1 contains at least one permanent magnet 3, a camera 6 that includes a lens 4 with a CCD sensor 5, and an antenna 8 for communication by radio with a remote station (not shown) outside of the patient 11.

In FIG., 1 the capsule 1 is shown three times, namely at different times T1, T2 and T3. At time T1, the patient 11 has just swallowed the capsule 1, which is why the capsule is situated on the path through an esophagus 28 in the direction of a stomach 30. At time T1, the capsule 1 may still be inactive if a gastrointestinal tract is to be investigated.

At time T2, the capsule 1 has reached the stomach 30. Examinations are carried out in the stomach 30. The direction of movement and speed of movement of the capsule 1, for example, are controlled by application of a force F and a torque M onto the capsule 1 using the magnetic coil system 13, which interacts with the permanent magnet 3. During this process, the camera 6 permits navigation by sight.

After time T2, the capsule 1 is navigated by sight through a pyloric orifice 40 and through a duodenum 42 as far as a small intestine 44. In the small intestine 44, the capsule 1 is depicted once again at time T3. Particularly on a path through the pyloric orifice 40, the duodenum 42 and the small intestine 44, obstructions of the capsule 1 may result due to friction against the walls or the capsule 1 becoming stuck in the gastrointestinal tract before the investigation has been completed and the capsule 1 is egested naturally from the patient 11 in the direction of an arrow 46. The present embodiments may enable the obstructions to be overcome more effectively. In one embodiment, an additional, brief movement (e.g., vibration or oscillation) of the capsule 1 is generated from outside. The additional movement supports the magnetic forces used for advancing the capsule 1 by effecting, for example, a breaking away from an organ wall. In one embodiment, movement is generated by changing a length of an exterior shell of the capsule 1.

In the embodiments described below, the additional movement is vibration for clarity of illustration. However, other additional movement of the capsule 1 may be provided in alternative embodiments.

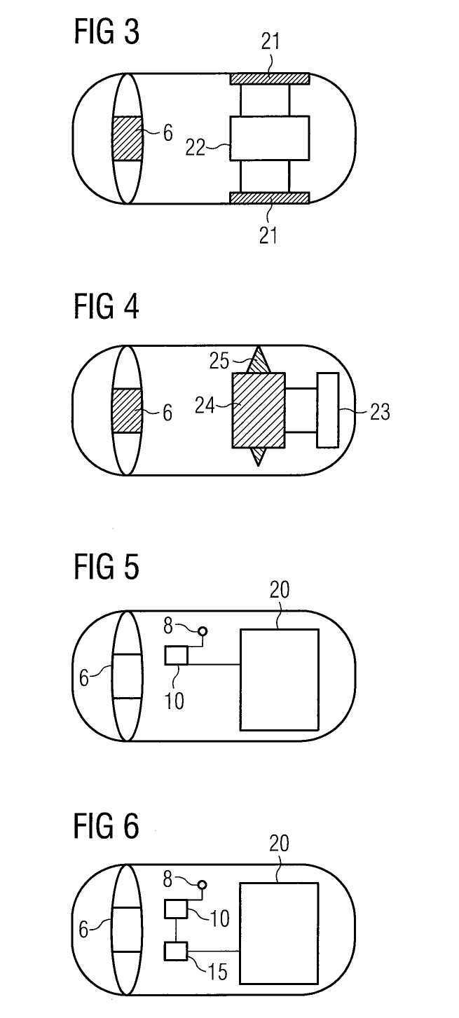

In one embodiment, the vibrations are generated using a device for generating vibrations that is contained in the capsule 1. Embodiments of the device for generating vibrations are shown in FIG. 3 and FIG. 4.

FIG. 3 shows an endoscopic capsule having, for example, ultrasonic resonators or transducers 21 for generating ultrasound. The ultrasonic resonators are driven using a circuit 22.

If an external controller detects that the capsule is blocked, the ultrasonic resonators are activated in accordance with one embodiment of a method illustrated below with reference to FIG. 5 and FIG. 6. As a result of the interaction of the ultrasonic resonator waves with the walls of the organ (e.g., intestine) in which the capsule is located, the capsule is set into motion until the blockage has been overcome.

FIG. 4 schematically illustrates one embodiment of the device for generating vibrations. A circuit 23 is connected to a coil 24, which surrounds a bobbin or coil carrier 25. If the capsule becomes blocked or gets stuck, the circuit 23 is supplied with energy according to one of the above-mentioned methods. By reversal of the polarity of the coil 24, vibrations are induced in the bobbin 25, and as a result, the capsule vibrates. This manner of operation is related to that of a doorbell or door chime, which is actuated using a relay.

In one embodiment not shown in the figures, a type of wobble-plate motor or unbalanced motor is arranged in the capsule, the motor serving to set the capsule into motion using internal forces acting asymmetrically.

In one embodiment, an outer shell of the capsule 1 is configured to undergo a change in length or shape induced by magnetostriction or electrostriction. In the event of problems in advancing the capsule, an electric or magnetic field is applied to change the shape. As a result of the change in shape, external forces (e.g., friction, normal advancement, gravitational force) come into play at other points of the capsule 1. Accordingly, a movement is generated, which counteracts obstructions during the advancement of the capsule.

FIG. 5 and FIG. 6 show two different embodiments for supplying energy to generate vibrations. For each embodiment, the figures show a device 20 for generating vibrations, an antenna 8, a receiver 10 for electromagnetic radiation and a camera 6.

According to one embodiment shown in FIG. 5, electromagnetic radiation received by the antenna 8 is used directly for generating vibrations. The radiation is forwarded by the receiver 10 to the device 20 for generating vibrations, where the device 20 feeds, for example, a circuit as shown in FIG. 3 or FIG. 4.

In FIG. 6, an energy store 15 (e.g. a battery) is shown. In response to a signal received from the antenna 8 and the receiver 10, the supply of energy from the energy store 15 to the device 20 is activated in order to generate vibrations. In one embodiment, logic may be provided, which evaluates received signals and interprets a correspondingly formed signal as a command to generate vibrations.

In one embodiment, the capsule may be configured to enable the energy store 10 to be charged using irradiated electromagnetic waves during an examination without causing vibrations to be triggered. The vibrations are dependent on an associated trigger signal.

In one embodiment, the duration of the vibrations may be limited. The duration of the vibrations may be limited, for example, by supplying the device with energy for the purpose of generating vibrations only for a desired time period. In one embodiment having an energy store as shown, for example, in FIG. 6, a timer or time recorder may be provided, which starts to run in response to the trigger signal for the vibrations. After the timer has timed out, the energy supply to the device for generating vibrations is interrupted again. After the limited time in which the capsule vibrates, the capsule is subject only to the influence of magnetic forces and may be navigated by the magnetic forces more effectively than if other movements (e.g., vibration) were to be superimposed on the navigation movements. The extraction of energy from the energy store is limited. In one embodiment, a termination of the vibrations or the energy supply required for the vibrations may be provided using an externally transmitted control signal.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A capsule for endoscopic examinations comprising:
a device for advancing the capsule through an organ under investigation;
a receiving system for electromagnetic radiation; and
a device for generating movements of the capsule to reduce edge friction impeding the advancement of the capsule, wherein the capsule is configured to activate the device for generating movements of the capsule using irradiation of the electromagnetic radiation.

2. The capsule as claimed in claim 1, wherein the device for advancing the capsule through the organ is a magnet.

3. The capsule as claimed in claim 1, wherein the device for generating movements of the capsule comprises an ultrasonic resonator.

4. The capsule as claimed in claim 1, wherein the device for generating movements of the capsule is implemented such that the capsule is made of a material in which a change in length is effected in the course of magnetostriction or electrostriction in order to generate movements, and a magnetic field or an electric field required for inducing the change in length of the material is applied.

5. The capsule as claimed in claim 1, wherein the device for generating movements of the capsule is supplied with energy by the irradiation of electromagnetic radiation, thereby activating the device.

6. The capsule as claimed in claim 5, wherein the irradiation of electromagnetic radiation for activation is used directly as an energy supply for the device for generating movements of the capsule.

7. The capsule as claimed in claim 5, further comprising an energy store, wherein the irradiation of electromagnetic radiation for activation is a signal to supply energy from the energy store to the device for generating movement of the capsule.

8. The capsule as claimed in claim 7, wherein the energy store is configured to be charged by energy transmitted wirelessly from an external source.

9. The capsule as claimed in claim 1, wherein the device for generating movements of the capsule is deactivated by a signal transmitted to the capsule from outside the capsule.

10. A method for assisting the advancement of an endoscopic capsule through organs, the method comprising:
irradiating a receiving system of the capsule with electromagnetic radiation to initiate a movement in addition to the advancement of the capsule,
wherein the irradiating the receiving system triggers an activation of a device for generating movements of the capsule to reduce edge friction impeding the advancement of the capsule.

11. The method as claimed in claim 10, wherein the movement is a vibration, pulsation, oscillation or a change in length of the capsule.

12. The method as claimed in claim 10, wherein the irradiating the receiving system with electromagnetic radiation supplies the device for generating movements of the capsule with energy, thereby activating the device.

13. The method as claimed in claim 12, wherein the electromagnetic radiation irradiated for activation is used directly as an energy supply for the device for generating movements of the capsule.

14. The method as claimed in claim 12, wherein the capsule comprises an energy store, and the electromagnetic radiation irradiated for activation is a signal to supply energy to the device from the energy store.

15. A capsule as claimed in claim 14, wherein the energy store is charged by energy transmitted wirelessly from an external source.

16. The capsule as claimed in claim 1, wherein the device for generating movements of the capsule comprises a bobbin arranged in a coil.

17. The capsule as claimed in claim 1, wherein the device for generating movements of the capsule comprises an unbalanced motor.

18. The capsule as claimed in claim 2, wherein the device for generating movements of the capsule is implemented such that the capsule is made of a material in which a change in length is effected in the course of magnetostriction or electrostriction in order to generate movements, and a magnetic field or an electric field required for inducing the change in length of the material is applied.

19. The capsule as claimed in claim 7, wherein the device for generating movements of the capsule is deactivated by a signal transmitted to the capsule from outside the capsule.

20. The method as claimed in claim 11, wherein the irradiating the receiving system with electromagnetic radiation supplies the device for generating movements of the capsule with energy, thereby activating the device.
